(19) United States
(12) Patent Application Publication
(10) Pub. No.: US 2024/0261338 A1
(43) Pub. Date: Aug. 8, 2024

(54) PHARMACEUTICAL COMPOSITION COMPRISING CHORION EXTRACT-DERIVED EXOSOMES AS ACTIVE INGREDIENT FOR PROMOTING OSTEOGENESIS

(71) Applicant: KOREA UNIVERSITY RESEARCH AND BUSINESS FOUNDATION, Seoul (KR)

(72) Inventors: Jae Jun SONG, Seoul (KR); Yoon Young GO, Seoul (KR)

(73) Assignee: KOREA UNIVERSITY RESEARCH AND BUSINESS FOUNDATION, Seoul (KR)

(21) Appl. No.: 18/558,811

(22) PCT Filed: May 2, 2022
(86) PCT No.: PCT/KR2022/006238

§ 371 Date: Nov. 3, 2023

(30) Foreign Application Priority Data
May 3, 2021 (KR) 10-2021-0057205

(51) Int. Cl. A61K 35/50; A61K 47/02; A61P 19/08

(52) U.S. Cl. CPC A61K 35/50; A61K 47/02; A61P 19/08

(57) ABSTRACT

The present invention relates to a pharmaceutical composition comprising exosomes within a chorion extract as an active ingredient for promoting osteogenesis and preventing and treating bone diseases and, more specifically, to excellent osteogenesis promotion efficacy of exosomes within a chorion extract prepared by extracting the placental chorion.

### TECHNICAL FIELD

The present disclosure relates to a pharmaceutical composition including exosomes within a chorion membrane extract as an active ingredient for promoting osteogenesis and, more specifically, to excellent osteogenesis promotion efficacy of exosomes within a chorion membrane extract prepared by extracting the placental chorion membrane.

### BACKGROUND ART

The amnion membrane is the innermost membrane constituting the placenta, and serves to protect the fetus from external pathogens by surrounding the amniotic fluid and the fetus and create an environment in which the fetus may grow and develop normally by maintaining biohomeostasis of the amniotic fluid and the fetus. In 1910, Davis first used the amnion membrane as a biomaterial (Davis J W et al., Johns Hopkins HOSP REP, 15; 307, 1910), and the amnion membrane has been widely used for the purpose of wound healing after burn, wound and ophthalmology surgery (Faulk W P et al., The Lancet, 315(8179):1156-1158, 1980, Mohammadi A A et al., Burns, 39(2):349-353, 2013, Dua H S et, al., Survey of Ophthalmology, 49(1):51-77, 2004). The amnion membrane is known to include an epidermal growth factor (EGF), a fibroblast growth factor (FGF), a transforming growth factor (TGF-b), a nerve growth factor (NGF), a hepatic growth factor (HGF), etc., and consists of an extracellular substrate composed of collagen and epithelial cells in itself and contains anti-inflammation and anti-bacterial ingredients and angiogenic modulatory properties (Hao Y et al., Cornea, 19(3):348-352, 2000).

Exosomes are known as external vesicles, extracellular vesicles (EVs), secretive microvesicles, etc., and are nano-sized vesicles which are secreted from cells into an external environment for intracellular information exchange. It was known that the exosomes include various substances such as proteins, lipids, nucleic acids, and metabolites that show biological activity to play a role in communication between cells as a medium that carries these functional vesicles, and recently, it has been known that miRNA in the exosomes performs important functions in vivo (Yez-M M et al., J Extracel Vesic. 2015:4:27066, Colombo M et al., Ann Rev Cell Dev Biol. 2014:30:25589, Valadi H et al., Nat Cell Biol. 2007:9(6):6549). The exosomes are found even in various body fluids and used even for diagnosis of diseases, and may be relatively and stably prepared and stored to be developed as diagnosis and treatment agents.

Bone that supports the soft tissue and weight of the human body and surrounds the internal organs to protect the internal organs from external shocks is one of important parts of the human body that not only structurally supports muscles and organs, but also stores substances such as internal calcium or other essential minerals, that is, phosphorus or magnesium. The full-grown adult bones are maintained in balance while continuously repeating generation and resorption processes of removing old bones and replacing the old bones with new bones, which is called bone remodeling (Yamaguchi A. et al., Tanpakushitsu Kakusan Koso., 50(6Suppl):664-669, 2005). Such a cycle of bones is required for restoring the microscopic damage of the bones caused by growth and stress and maintaining the functions thereof. In adults, about 10 to 30% of the skeleton is re-formed each year through the remodeling of bone resorption-osteogenesis.

The bone remodeling involves osteoblasts that generate bones and osteoclasts that destroy bones, and they are closely associated with each other to maintain the homeostasis of the bones. For example, the osteoblasts maintain the bone homeostasis in the body by controlling the differentiation of osteoclasts that are responsible for bone resorption through the secretion of substances such as receptor activator of nuclear factor-kligand (RANKL), and osteoprotegerin (OPG) as an inductive receptor thereof. With respect to treatment of bone diseases involved when the homeostasis of bones is not maintained by physical effects, hormonal system, etc. and in the case of bone tissue damage, in the past, studies have been conducted based on only metabolic abnormality of bone minerals, such as calcium and phosphorus, and the identification of the mechanism was not advanced. In general, a diet containing calcium is recommended for the treatment and prevention of osteoporosis, and administration of estrogen or vitamin D is recommended for women in menopause. In addition, bisphosphonate series such as Fosamax (ingredient name: alendronate) and Actonel (ingredient name: risedronate) are new alternative therapeutic agents as bone resorption inhibitors that inhibit osteoclasts and induce death. However, it was known that calcium reinforcements, which are widely used as bone disease therapeutic agents, inhibit the secretion of parathyroid hormones and prevent reduced bone mass due to bone resorption, but the individual difference in maintenance of bone mass is severe (Heandy R. P. principles of bone biology, Academic press, 1007-1017, 1996). In addition, hormonal therapy using estrogen or calcitonin has been reported to increase bone density and reduce the occurrence of rectal cancer, but reported to have side effects such as breast cancer, myocardial infarction, and venous thrombosis (Nelson, H. D et al., JAMA, 288:872-881, 2002; Lemay, A., J. Obstet. Bynaecol. Can., 24:711-7152-3). In addition, in the case of bisphosphonate preparations, in recent years, cases in which necrosis of the jaw bone, severe atrial fibrillation, incapacitation of bones or joints, or pain in musculoskeletal occurs in taking patients are increasing year by year (Coleman R E., Br J Cancer, 98:1736-1740(2008). Accordingly, the development of a new therapeutic agent for bone diseases that may effectively inhibit bone resorption even while having few side effects has been required.

Conventionally, the efficacy of promoting differentiation and osteogenesis of osteoblasts in exosomes within a chorion membrane extract has never been disclosed, and the exosomes of the chorion membrane extract are not used for diseases requiring osteoanagenesis. Therefore, the present inventors separated and analyzed exosomes within a chorion membrane extract, confirmed that osteoanagenesis promotion efficacy was excellent when treating the exosomes within the chorion membrane extract other than the chorion membrane extract when inducing bone differentiation of osteoblasts, and provided the present disclosure.

### PRIOR ARTS

Korean Patent Registration No. 10-1701227
Korean Patent Registration No. 10-1824521

### DISCLOSURE OF THE INVENTION

### Technical Goals

An aspect of the present disclosure is to provide a pharmaceutical composition including chorion membrane extract-derived extracellular vesicles as an active ingredient for promoting osteogenesis and preventing or treating bone diseases.

However, technical goals to be achieved are not limited to those described above, and other goals not mentioned above are clearly understood by one of ordinary skill in the art from the following description.

### Technical Solutions

According to an embodiment of the present disclosure, there is provided a pharmaceutical composition for promoting osteogenesis including chorion membrane extract-derived extracellular vesicles as an active ingredient.

According to another embodiment of the present disclosure, there is provided a pharmaceutical composition for preventing or treating bone diseases including chorion membrane extract-derived extracellular vesicles as an active ingredient.

According to an aspect, the bone diseases may be one selected from the group consisting of bone defects, osteoporosis, osteoporotic fracture, diabetic fractures, fracture nonunion, osteogenesis imperfecta, osteomalacia and fractures resulting therefrom, osteogenesis disorders, degenerative bone diseases, or malocclusion.

According to an aspect, the extracellular vesicles may be at least one selected from the group consisting of exosomes, microvesicles, or microparticles.

According to an aspect, the extracellular vesicles may have a concentration of 1×10¹⁰ to 1×10¹² particles/well (herein, the well means 24 wells).

According to an aspect, the extracellular vesicles may have an average particle size of 100 nm to 150 nm.

According to an aspect, the composition may be mixed and used with a hydroxyapatite/β-tricalcium phosphate (HA/bTCP) scaffold.

According to an aspect, the composition may be formulated into powders, granules, tablets, capsules, suspensions, emulsions, syrups, aerosols, external preparations, suppositories, or sterile injectable solutions.

### Effects

According to the present disclosure, the chorion membrane extract-derived extracellular vesicles may promote differentiation of osteoblasts and osteogenesis.

Further, the chorion membrane extract-derived extracellular vesicles of the present disclosure may have excellent mineral deposition compared to a chorion membrane extract.

Further, the chorion membrane extract-derived extracellular vesicles of the present disclosure show an excellent osteogenesis effect (calcium deposition evaluation) compared to using a chorion membrane extract or a chorion/amnion membrane extract.

It should be understood that the effects of the present disclosure are not limited to the effects described above, but include all effects that may be deduced from the detailed description of the present disclosure or configurations of the disclosure described in claims.

### BRIEF DESCRIPTION OF DRAWINGS

FIG. 1, that is, FIGS. 1A to 1D illustrate results of isolating and analyzing exosomes within an amnion membrane extract and a chorion membrane extract.

FIG. 2 illustrates the ALP activities of the amnion membrane extract and the chorion membrane extract.

FIG. 3, that is, FIGS. 3A and 3B illustrate results of Alizarin Red S staining for a chorion membrane extract and exosomes within the extract at day 14 after differentiation, respectively.

FIG. 4, that is, FIGS. 4A and 4B illustrate ALP activation and calcium deposition as results of comparing and experimenting osteoanagenesis efficacy of an amnion membrane, an amnion/chorion membrane extract, and a chorion membrane extract.

### BEST MODE FOR CARRYING OUT THE INVENTION

Hereinafter, embodiments will be described in detail with reference to the accompanying drawings. Like reference numerals presented in each drawing indicate like elements.

Various modifications may be made to embodiments to be described below. Embodiments to be described below are not intended to be limited to aspects and should be understood to include all modifications, equivalents, and substitutes thereof.

Terminologies used herein are used only to describe specific embodiments, and are not intended to limit the embodiments. The singular expression includes the plural expression unless the context clearly dictates otherwise. In the present specification, it should be understood that term “comprising” or “having” indicates that a feature, a number, a step, an operation, a component, a part or the combination thereof which is implemented is present, but does not exclude a possibility of presence or addition of one or more other features, numbers, steps, operations, components, parts or combinations thereof, in advance.

Unless otherwise contrarily defined, all terms used herein including technological or scientific terms have the same meanings as those generally understood by a person with ordinary skill in the art to which embodiments pertain. Terms which are defined in a generally used dictionary should be interpreted to have the same meaning as the meaning in the context of the related art, and are not interpreted as ideal or excessively formal meanings unless otherwise defined in the present application.

In addition, in the description with reference to the accompanying drawings, like components regardless of reference numerals designate like reference numerals and a duplicated description thereof will be omitted. In describing the embodiments, a detailed description of related known technologies will be omitted if it is determined that they unnecessarily make the gist of the embodiments unclear.

According to an embodiment of the present disclosure, there is provided a pharmaceutical composition for promoting osteogenesis including chorion membrane extract-derived extracellular vesicles as an active ingredient. The osteogenesis promotion includes osteogenesis promotion of osteoblasts during implantation.

The term ‘extracellular vesicles’ used herein refer to nano-sized vesicles secreted by cells into an external environment, and include exosomes, microparticles, micro-endoplasmic reticlum, and microvesicles.

The chorion membrane extract-derived extracellular vesicles may be prepared by a preparing method including isolating and extracting the chorion membrane from the human placenta; and treating the chorion membrane extract with an Exoquick solution and centrifuging to collect extracellular vesicles.

The osteogenesis promotion may be performed by promoting differentiation of osteoblasts by the chorion membrane extract-derived extracellular vesicles.

According to another embodiment of the present disclosure, there is provided a pharmaceutical composition for preventing or treating bone diseases including chorion membrane extract-derived extracellular vesicles as an active ingredient.

The term ‘bone diseases’ used herein may be one selected from the group consisting of bone defects, osteoporosis, osteoporotic fracture, diabetic fractures, fracture nonunion, osteogenesis imperfecta, osteomalacia and fractures resulting therefrom, osteogenesis disorders, degenerative bone diseases, or malocclusion, but are not limited thereto.

According to an aspect, the extracellular vesicles may be at least one selected from the group consisting of exosomes, micro-endoplasmic reticlum, microvesicles, or microparticles, but are not limited thereto, and as defined above, as membranous vesicles secreted by cells to the external environment, may include various substances showing biological activity such as lipids, nucleic acids, proteins, and metabolites.

According to an aspect, the extracellular vesicles may have a concentration of 1×10¹⁰ to 1×10¹² particles/well. Desirably, the extracellular vesicles may have a concentration of 1×10¹² particles/well.

At the concentration, the extracellular vesicle composition in the chorion membrane extract may show high ALP activity and mineral deposition, and have excellent osteoblast differentiation promotion and osteogenesis promotion efficacies.

The ALP activity is an indicator of the early differentiation and osteogenesis of osteoblasts and indicates the degree to which an alkaline phosphatase (ALP) enzyme is activated. Desirably, the ALP activity may be measured at 405 nm using an ALP kit from Anaspec, Inc., Fermont, Canada.

The mineral deposition may be evaluated by Alizarin Red S staining, which is widely used as a marker for mid-to-late differentiation of osteoblasts. Desirably, the mineral deposition may be measured using a staining solution from Millipore according to the manufacturer's instructions.

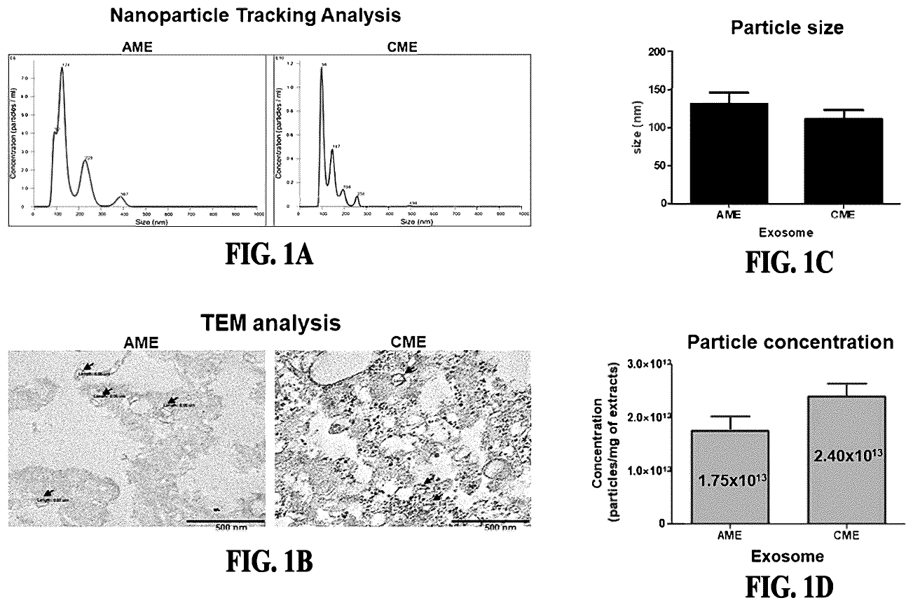

According to an aspect, the extracellular vesicles may have an average particle size of 100 nm to 150 nm. When describing the sizes of exosomes within the amnion membrane and chorion membrane extracts in FIG. 1C, it may be confirmed that the sizes of the exosomes within the chorion membrane extract are smaller than those of the exosomes within the amnion membrane extract.

According to an aspect, the composition may be mixed and used with a hydroxyapatite/β-tricalcium phosphate (HA/bTCP) scaffold. The HA/bTCP is used as an osteoanagenesis composite scaffold, and the like, and when used in the composition, an osteoanagenesis effect may be increased.

According to an aspect, the composition may be formulated into powders, granules, tablets, capsules, suspensions, emulsions, syrups, aerosols, external preparations, suppositories, or sterile injectable solutions.

The pharmaceutical composition of the present disclosure may further include a pharmaceutically acceptable carrier. These carriers are commonly used in formulations and include lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methylcellulose, methylhydroxybenzoate, propylhydroxybenzoate, talc, magnesium stearate, and mineral oils, but are not limited thereto. The pharmaceutical composition may further include a lubricant, a wetting agent, a sweetening agent, a flavoring agent, an emulsion, a suspension, a preservative, and the like, in addition to the ingredients.

The pharmaceutical composition is desirably administered parenterally, and may be administered, for example, by using intravenous administration, intraperitoneal administration, intratumoral administration, intramuscular administration, subcutaneous administration, or local administration.

A suitable dose of the pharmaceutical composition varies depending on factors, such as a formulation method, an administration method, age, weight, sex, and disease condition of a patient, food, an administration time, an administration route, an excretion rate, and response sensitivity, and an ordinarily skilled physician may easily determine and prescribe a dose effective for desired treatment.

The pharmaceutical composition may be formulated by using a pharmacologically acceptable carrier and/or excipient according to a method that may be easily performed by those skilled in the art to be prepared in a unit dose form or prepared by introduction into a multi-dose container. In this case, the formulations may also be the forms of solutions, suspensions, or emulsions in oils or aqueous media or the forms of excipients, powders, granules, tablets or capsules, and may additionally include dispersants or stabilizers.

Hereinafter, the present disclosure will be described in more detail with reference to Examples. The following Examples are described for the purpose of illustrating the present disclosure, and the scope of the present disclosure is not limited thereto.

### Example 1: Preparation of Chorion Membrane Extract

The placenta was cut around the umbilical cord, and the amnion membrane was carefully peeled from the placenta to be isolated and collected into the amnion membrane and the chorion membrane. The collected chorion membrane was washed in PBS or physiological saline added with 1% penicillin/streptomycin antibiotics. After removing the mucus and blood from the chorion membrane, the chorion membrane was pulverized using a homogenizer. A buffer solution was added at a ratio of 1:1 of the mass (g) of the chorion membrane and the volume (mL) of PBS, and proteins were released at 4° C. for 16 hours (overnight). After the proteins were released using an ultrasonicator, the extract was centrifuged using a centrifuge to collect a supernatant, and then filtered through a syringe and stored at −80° ° C. The chorion membrane extract was used in an appropriate amount in an experiment by measuring the concentration of proteins.

### Example 2: Identification of Exosomes within Chorion Membrane Extract and Amnion Membrane Extract

1 mL each of a chorion membrane extract and an amnion membrane extract was mixed with 0.2 mL of an Exoquick solution (manufactured by SBI) and exosomes were isolated at 4° C. for 16 hours (overnight), and then collected using a centrifuge. The isolated exosomes were diluted 1/10 or 1/20 and analyzed for size and concentration using NTA equipment, and double phospholipid-shaped exosomes were identified using TEM equipment.

According to FIG. 1C, it could be seen that the sizes of exosomes within amnion membrane extract (AME) and chorion membrane extract (CME) were in the range of 100 nm to 150 nm, but the sizes of the exosomes within the chorion membrane extract were smaller. Meanwhile, as a result of confirming the number of exosomes per mg of each extract, it was confirmed that the concentration (2.40×10¹³) of exosomes within the chorion membrane extract was much higher than that of exosomes within the amnion membrane extract.

### Example 3: Measurement of Osteogenesis Effect of Chorion Membrane Extract

In the present disclosure, MG-63 (Korea Cell Line Bank, KCLB), a human osteosarcoma cell line, was used in an in vitro osteogenesis experiment, and the cell line was known to be similar to osteoblasts. Cells were cultured under culture conditions of a Dulbecco's modified Eagle's medium (DMEM) containing 10% (v/v) fetal bonine serum (FBS) and 1% (w/v) penicillin/streptomycin and a humid environment at 37° C. and 5% CO₂.

### Example 3-1: Measurement of ALP Activity

An experiment was conducted to measure ALP enzyme activity, an osteogenesis indicator that appeared in the early stages of osteogenesis. To confirm an ALP effect of exosomes within the chorion membrane extract, in an osteogenesis induction medium (OIM), and Examples treated with an amnion membrane extract, exosomes within the amnion membrane extract, a chorion membrane extract, and exosomes within the chorion membrane extract in the medium, respectively, the ALP activities were measured. At this time, the exosomes within the chorion membrane extract and the amnion membrane extract were treated at 1×10¹⁰ particles/well, 1×10¹¹ particles/well, and 1×10¹² particles/well (based on 24 well), respectively. In the case of the chorion membrane extract, MG-63 cells were treated with the chorion membrane extract at a concentration of 200 μg/well in the osteogenesis induction medium.

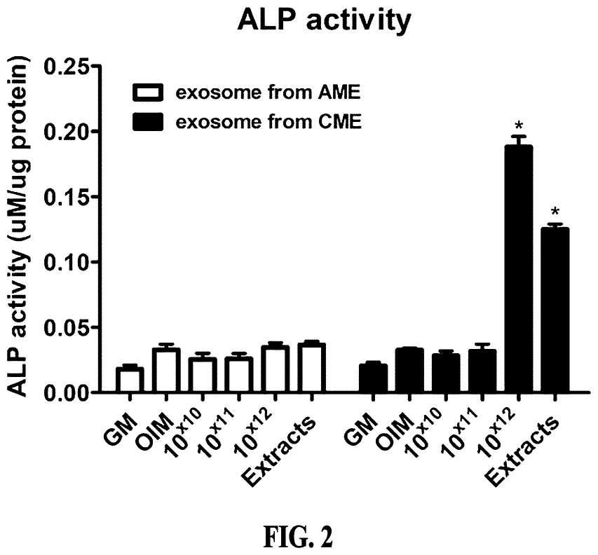

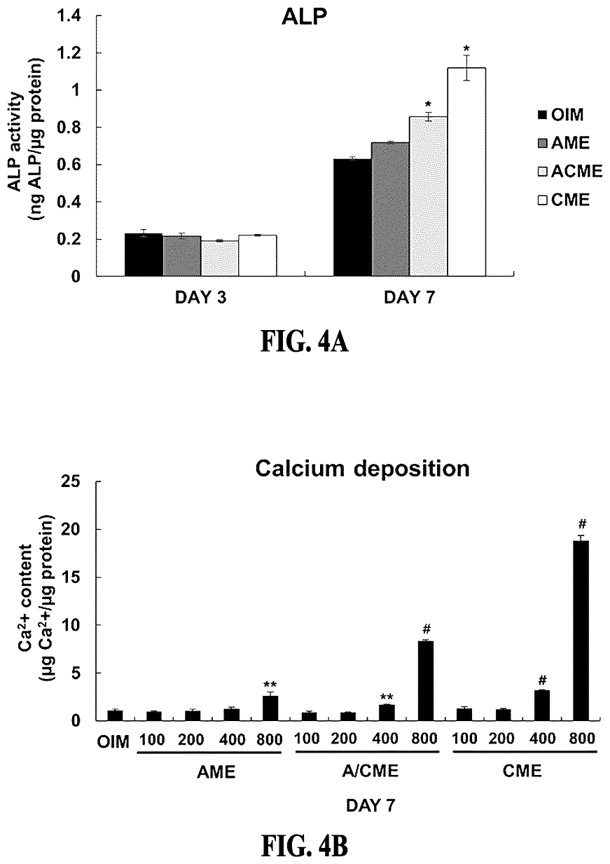

Specifically, samples were taken on day 3 and day 7, washed twice with cold PBS buffer, and then lysed with a cell lysis buffer containing 0.1% Triton X-10. The lysed cells were centrifuged at 4° C. and 13000 rpm for 30 minutes, and then the supernatant was recovered and quantified using a Bradford method, and ALP activity of the same amount of protein was measured at 405 nm using an ALP kit (Anaspec, Inc., Fermont, Canada) according to the manufacturer's instructions, and the results were illustrated in FIGS. 2 and 4A. According to FIG. 2, it was confirmed that ALP activity was not shown in an untreated osteogenesis induction medium (OIM), an amnion membrane extract, and exosomes within the amnion membrane extract, and a group treated with exosomes within the chorion membrane extract showed higher ALP activity than other experimental groups (*P<0.05). In particular, in the case of 1×10¹² particles/well, very high ALP activity was observed compared to other experimental groups.

### Example 3-2: Evaluation of Alizarin Red S Staining

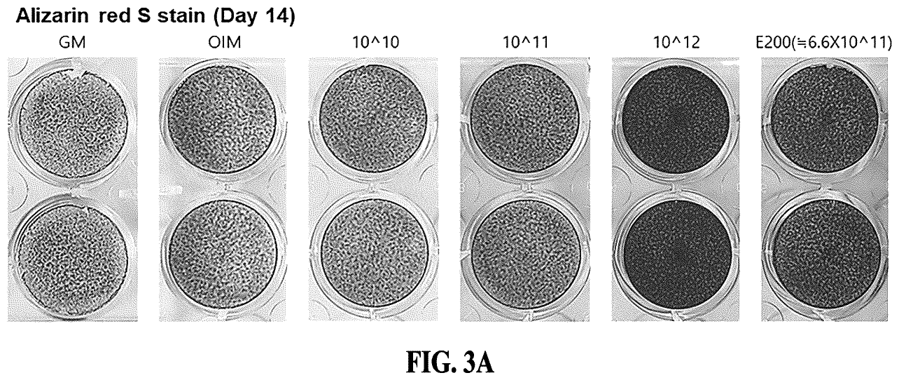

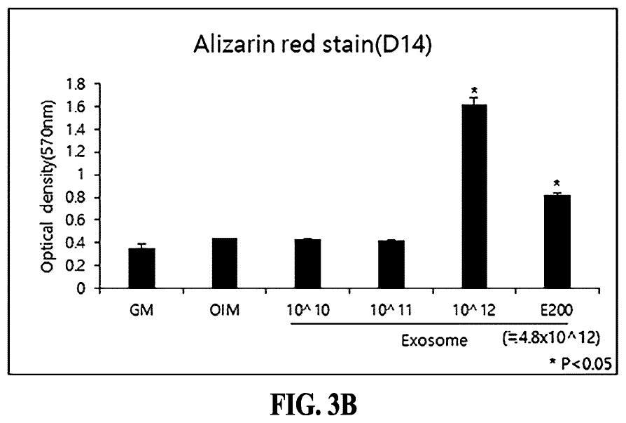

Alizarin Red S staining evaluation is widely used as a mid-to-late marker during differentiation of osteoblasts. To compare the mineral deposition of the chorion membrane extract and exosomes within the chorion membrane extract, an Alizarin Red S staining evaluation experiment was performed. Equally, MG-63 cells were differentiated in the osteogenesis induction medium (OIM), and treated with 200 μg/well each of the chorion membrane extract (experimented by selecting the concentration based on a conventional experimental result that 200 μg/well of the chorion membrane extract had the best osteoanagenesis effect) and the exosomes within the chorion membrane extract at 1×10¹⁰ particles/well, 1×10¹¹ particles/well, and 1×10¹² particles/well, respectively, and then the mineral content was measured after 14 days of differentiation. The mineral content was measured using an Alizarin Red S staining solution (Millipore) according to the manufacturer's instructions, and the results were shown in FIGS. 3A and 3B, respectively. According to FIG. 3B, it was confirmed that the mineral deposition-inducing effect was very excellent in the group treated with exosomes within the chorion membrane extract compared to the group treated with the chorion membrane extract (*P<0.05, Day 14).

According to Examples 3-1 and 3-2, 200 μg of the chorion membrane extract included 4.8×10¹² particles/well of the exosomes, but the ALP activity and the mineral deposition effect were rather lower than the group treated with the exosomes within the chorion membrane extract of 1×10¹² particles/well. Meanwhile, from these results, it may be confirmed that the exosomes within the chorion membrane extract have an excellent osteogenesis promotion effect and are a substance with a better osteogenesis effect than the chorion membrane extract itself.

### Example 3-3: Evaluation of ALP Activity and Calcium Deposition

In order to identify and verify extracts containing osteogenesis promotion efficacy, ALP activity and calcium deposition were compared and evaluated during bone differentiation of osteoblasts when treated with an amnion membrane extract (AME), an amnion/chorion membrane extract (ACME), and a chorion membrane extract (CME). Equally, after treated with the amnion membrane extract, the amnion/chorion membrane extract, and the chorion membrane extract during MG-63 cell differentiation in the osteogenesis induction medium, respectively, on day 7 of differentiation, ALP activation and calcium deposition content were measured.

According to FIGS. 4A and 4B, it was confirmed that the chorion membrane extract had a much better osteogenesis promotion effect than the amnion membrane extract, and the osteogenesis promotion efficacy was included in the chorion membrane extract.

As described above, although the embodiments have been described by the restricted embodiments and the drawings, various modifications and variations may be made from the above description by those skilled in the art. For example, even if the described techniques are performed in a different order from the described method, and/or components described above are coupled or combined in a different form from the described method, or replaced or substituted by other components or equivalents, an appropriate result may be achieved. Therefore, other implementations, other embodiments, and equivalents to the appended claims fall within the scope of the claims to be described below.

1. A pharmaceutical composition for promoting osteogenesis comprising chorion membrane extract-derived extracellular vesicles as an active ingredient.

2. A pharmaceutical composition for preventing or treating bone diseases comprising chorion membrane extract-derived extracellular vesicles as an active ingredient.

3. The pharmaceutical composition for preventing or treating bone diseases of claim 2, wherein the bone diseases are one selected from the group consisting of bone defects, osteoporosis, osteoporotic fracture, diabetic fractures, fracture nonunion, osteogenesis imperfecta, osteomalacia and fractures resulting therefrom, osteogenesis disorders, degenerative bone diseases, or malocclusion.

4. The pharmaceutical composition of claim 1, wherein the extracellular vesicles are at least one selected from the group consisting of exosomes, microvesicles, or microparticles.

5. The pharmaceutical composition of claim 1, wherein the extracellular vesicles have a concentration of 1×10¹⁰ to 1×10¹² particles/well.

6. The pharmaceutical composition of claim 1, wherein the extracellular vesicles have an average particle size of 100 nm to 150 nm.

7. The pharmaceutical composition of claim 1, wherein the composition is mixed and used with a hydroxyapatite/β-tricalcium phosphate (HA/bTCP) scaffold.

8. The pharmaceutical composition of claim 1, wherein the composition is formulated into powders, granules, tablets, capsules, suspensions, emulsions, syrups, aerosols, external preparations, suppositories, or sterile injectable solutions.

9. The pharmaceutical composition of claim 2, wherein the extracellular vesicles are at least one selected from the group consisting of exosomes, microvesicles, or microparticles.

10. The pharmaceutical composition of claim 2, wherein the extracellular vesicles have a concentration of 1×10¹⁰ to 1×10¹² particles/well.

11. The pharmaceutical composition of claim 2, wherein the extracellular vesicles have an average particle size of 100 nm to 150 nm.

12. The pharmaceutical composition of claim 2, wherein the composition is mixed and used with a hydroxyapatite/β-tricalcium phosphate (HA/bTCP) scaffold.

13. The pharmaceutical composition of claim 2, wherein the composition is formulated into powders, granules, tablets, capsules, suspensions, emulsions, syrups, aerosols, external preparations, suppositories, or sterile injectable solutions.

14. The pharmaceutical composition of claim 3, wherein the extracellular vesicles are at least one selected from the group consisting of exosomes, microvesicles, or microparticles.

15. The pharmaceutical composition of claim 3, wherein the extracellular vesicles have a concentration of 1×10¹⁰ to 1×10¹² particles/well.

16. The pharmaceutical composition of claim 3, wherein the extracellular vesicles have an average particle size of 100 nm to 150 nm.

17. The pharmaceutical composition of claim 3, wherein the composition is mixed and used with a hydroxyapatite/β-tricalcium phosphate (HA/bTCP) scaffold.

18. The pharmaceutical composition of claim 3, wherein the composition is formulated into powders, granules, tablets, capsules, suspensions, emulsions, syrups, aerosols, external preparations, suppositories, or sterile injectable solutions.
